(19) United States
(12) Patent Application Publication
(10) Pub. No.: US 2024/0042193 A1
(43) Pub. Date: Feb. 8, 2024

(54) ELECTROTHERAPY APPLIANCE FOR DETERMINING POSITIONS OF THE ELECTROTHERAPY APPLIANCE

(71) Applicant: KEYTEC GMBH, Diedorf (DE)

(72) Inventor: Ulrich BURR, Erftstadt (DE)

(21) Appl. No.: 18/269,011

(22) PCT Filed: Dec. 15, 2021
(86) PCT No.: PCT/EP2021/085896

§ 371 Date: Jun. 22, 2023

(30) Foreign Application Priority Data
Dec. 23, 2020 (EP) 20217018.9

(51) Int. Cl. A61N 1/04; A61N 1/40

(52) U.S. Cl. CPC A61N 1/0476; A61N 1/40

(57) ABSTRACT

An electrotherapy appliance for introducing a therapeutic electrical pulse into the skin of a patient includes a pulse source for outputting a pulse voltage and an output capacitor, which can be placed on the skin of the patient and is fitted with a first capacitor electrode connected to a first terminal of the pulse source and fitted with a second capacitor electrode connected to a second terminal of the pulse source, in order to convert the pulse voltage into the therapeutic pulse. A position sensor is provided for detecting a position of the output capacitor on the skin while the therapeutic pulse is being delivered.

### BACKGROUND AND SUMMARY OF THE INVENTION

Exemplary embodiments of the invention relate to an electrotherapy appliance and to a method for establishing a therapy protocol by means of an electrotherapy appliance.

Electrotherapy should ideally be performed in several sessions. The scope of the previous session should be followed up, e.g., in case of a follow-up session.

An electrotherapy appliance is known from RU 2004 136 418 A. With this appliance, the therapist must make a manual record of which parts of the body and in which way the therapy has been carried out so far. For this purpose, the therapist can use, for example, an anatomy drawing of a human being, in which the therapist marks the respective therapy paths and trigger points that have been treated with the electrotherapy appliance. This procedure is time-consuming and labor-intensive. At the same time, it is important to measure and note the skin reaction on the respective therapy path or trigger point, in the sense of saving it. The skin reaction to program-dependent therapy impulses is measured as the first pulse time and the number of zero crossings, which in turn are a measure of the skin impedance at precisely this point on the skin surface. The correlation of locally assignable and stored skin reactions and physiological well-being as well as the comparison with older therapy sessions or also experiences from similar cases can be used on the one hand for an objective evaluation of the current therapy success and on the other hand for an optimization of the treatment of subsequent sessions.

This invention lays the technical foundation to digitize the individual possibilities (determined by therapist and device) of the previously known electrotherapy according to RU 2004 136 418A, and thus to make them objectively optimizable and verifiable. This technical prerequisite makes it possible to objectify and automate subjective experiences of therapists on a digital level.

The use of position sensors is already known from fields of application that are remote from the application and thus foreign to the genus. EP 3 195 898 A1 discloses a device for vaginal remodeling. It comprises a disposable, sterilizable handpiece which is an elliptical or preferably cylindrical applicator suitable for insertion into the vagina, wherein at least one electrode is arranged either on the outer surface of the handpiece or on the inner surface of the handpiece. This electrode determines the insertion depth of the applicator into the handpiece. The device may further disclose other position sensors that determine the insertion depth of the overall device into a user. It is clear from the overall context of the document that this is not about position detection in a plane, such as along an area of skin, but rather an application in which something is inserted into the vagina. Such a method of determining the position of an insertion depth is therefore completely unsuitable for use in an electrotherapy appliance, such as RU 2004 136 418 A.

Exemplary embodiments of the invention are directed to improving the known electrotherapy appliance.

In accordance with one aspect of the invention, an electrotherapy appliance for introducing a therapeutic electrical pulse into the skin of a patient comprises a pulse source for outputting a pulse voltage; an output capacitor which can be placed on the skin of the patient and is fitted with a first capacitor electrode connected to a first terminal of the pulse source and fitted with a second capacitor electrode connected to a second terminal of the pulse source, in order to convert the pulse voltage into the therapeutic pulse.

According to the invention, the electrotherapy appliance comprises a position sensor for detecting a position of the output capacitor on the skin while the therapeutic pulse is being delivered.

The invention is based on the consideration that not only the skin can be treated with conventional electrotherapy appliances, the development of the therapy can be read out directly from the electrotherapy appliance, for example, based on the electrical properties of the skin. In principle, therapy protocols could be created in order to reliably document the patient's therapy, even to monitor its success or, in case of doubt, even to compare it with other therapy methods. However, the reliability and informative value of the therapy protocols essentially depends on how reliably the therapist reproducibly targets a specific region on the skin with the output capacitor, which in principle could still be solved by graphic auxiliary lines on the skin to be treated. However, the therapist usually has a large number of therapy points to be approached on the skin, which must be reliably kept apart if the therapy protocols are not to lose their significance.

This is where the invention engages with the idea of including position detection in the electrotherapy appliance. In this way, the therapist no longer needs to concentrate on which region on the skin he is applying the specified electrotherapy appliance. The position data can be collected reliably and without error by the position sensor, thus significantly increasing the informative value of the therapy protocols. The therapist can thus concentrate more on the actual therapy, which eliminates potential sources of error and makes work easier.

Finally, the therapy protocols can be generated not only over time, but also in local dependence on therapy pathways, which significantly increases the informative value of the collected therapy protocols.

Advantageously, not only the position in one dimension (i.e., an insertion depth) can be determined via the position sensor—as in EP 3 195 898 A1. This would not bring any advantage for an area of application which mainly concerns the skin surface, but it is preferably a dx−dy—i.e., a 2-dimensional-displacement sensor.

Particularly preferred in the present case is the use of an optical path sensor, which is also not preferred, for example, when inserting the sensor into dark body openings—as in the case of EP 3 195 898 A1.

In a further development of the disclosed electrotherapy appliance, the pulse source comprises an oscillating circuit with the output capacitor and a magnetically chargeable coil. A resonant frequency of the oscillating circuit, whether from a parallel circuit or a series circuit of the output capacitor and the coil, allows immediate conclusions to be drawn about the skin being treated because the skin changes the capacitance of the output capacitor. Together with the recorded position, very precise therapy protocols can therefore be created, which provide clear information about the course of treatment.

In order to magnetically charge the coil in a simple way, it can preferably be part of a transformer as a secondary coil, which is charged by a primary coil. The structure of the transformer used preferably does not allow galvanic separation between the primary and secondary coils, since both windings are connected at the upper end. This is advantageous and part of the concept of a bio-feedback function, in which a signal is generated to evaluate the therapy. In particular, the skin or tissue is always parallel to the secondary circuit, influences with its properties both the pre-pulse (charging) and the therapeutic pulse (discharging).

Charging or recharging in the context of the present invention can be understood, in particular, as a conversion into magnetic energy by the primary coil, which is stored in the magnetic field and transformer core (ferromagnetic material). When the current to the primary coil is switched off, the stored magnetic energy is converted back into electrical energy and released through the secondary coil via the skin.

In a particular further development, the specified electrotherapy appliance comprises a transmitting interface for transmitting the position of the output capacitor to a data processing device. This transmitting interface can in principle be of any design, i.e., wired or wireless. For a bandwidth- and energy-saving connection of the specified electrotherapy appliance to the data processing device, transmission via Bluetooth Low Energy is particularly suitable, because in this way other devices can also be connected to the data processing device in a space-, energy- and bandwidth-saving manner.

In a particularly preferred further development of the specified electrotherapy appliance, the transmitting interface is set up to transmit the position of the output capacitor together with a measured value describing the skin at the detected position. This measured value can be, for example, the previously mentioned resonant frequency of the oscillating circuit, but also the capacitance of the skin at the detected position. In this way, the property of the skin can be precisely resolved locally, which is a much improved aid to the therapist in evaluating the success of the treatment.

In another further development of the specified electrotherapy appliance, the position sensor is set up to incrementally detect the position of the capacitor on the skin. Even if the detection of the position can in principle be implemented absolutely, for example via a spatial position system, the incremental detection allows the therapist to define his own reference points, starting from which he wants to resolve the skin measurement locally. This allows the specified electrotherapy appliance to be used more intuitively.

In principle, the incremental position detection can be detected in any manner using spheres, acceleration sensors, non-contact distance sensors, or the like. In an additional further development of the disclosed electrotherapy appliance, the position sensor for incremental position detection is arranged to capture an image of the skin at regular intervals and to compare it with a previously captured image of the skin. This principle is already successfully used in computer mice for controlling a cursor on a screen. In contrast to the other incremental position detection systems mentioned, the movement of the output capacitor on the skin can be detected in space despite natural spherical irregularities on the surface of the patient. Spatial movement of the capacitor on the patient's skin due to the spherical unevenness is immediately detected as two-dimensional motion, eliminating the need for any coordinate transformation or the like.

In yet another further development of the specified electrotherapy appliance, the capacitor electrodes are formed as closed loops arranged concentrically to one another. In this way, a comparatively large air gap can be created between the two capacitor electrodes in the smallest possible space, whereby a high sensory sensitivity is achieved for the specified electrotherapy appliance.

In a particularly expedient further development of the specified electrotherapy appliance, the position sensor is arranged within the inner loop of the loops arranged concentrically to each other, preferably in the center thereof. Since the electric field between the two loops is built up in the air gap, the interior of the inner loop is practically field-free, analogous to a coaxial cable. In this way, the risk of electromagnetic interference is reduced or avoided altogether.

In yet another further development of the specified electrotherapy appliance, the output capacitor is held on a treatment head having a recess accommodating the position sensor. In this way, the output capacitor can be pressed flat onto the skin to be treated without the position sensor interfering during treatment.

In another further development of the specified electrotherapy appliance, the recess is closed with a cover. In this way, the position sensor can be protected from contamination, such as skin sweat or the like.

Furthermore, the electrotherapy appliance can have an acceleration sensor for determining the accelerations in at least two axes, in particular in all three axes, for validating the signal of the position sensor.

The electrotherapy appliance can preferably be designed as a hand-held device. Furthermore, the electrotherapy appliance, in particular in the design as a hand-held device, can be battery-operated and/or battery-powered.

Especially when the above-mentioned computer mouse principle is used for the position sensor, the cover should be of transparent design.

Furthermore, according to the invention, there is a method for creating a therapy protocol, which comprises determining positions of the electrotherapy appliance according to the invention on the skin, wherein a movement of the electrotherapy appliance relative to a predetermined starting point is detected, so that the positions are detected as relative positions relative to the starting position by the position sensor, wherein the positions are logged. The electrotherapy appliance can thereby also be used for distance measurement of individual skin points without emitting a therapeutic pulse.

Thus, according to the invention, the creation of a data provision and protocol creation is protected. The therapeutic treatment by the electrotherapy appliance itself can be carried out as described in the prior art.

In addition to the respective position, the therapy protocol can contain a measured value determined by the electrotherapy appliance about the condition of the skin, in particular at the time of the position measurement. The therapy protocol can thus contain the location, the time and the current as well as punctual measured value about the condition of the skin.

This measured value can be determined, in particular, from the parameters of a pulse voltage, for example as the value of a resonant frequency and/or as the value of the capacitance of the skin, wherein a resonant frequency is inversely proportional to a first pulse time. A first pulse time corresponds to half of a period duration. An inverse of the period time in turn corresponds to the resonant frequency. A number of zero crossings gives information about the temporal length of a damped oscillation determined by the local skin properties.

Based on the measured values, a certain patient-specific adjustment of the device settings can be made. In particular, this can also be varied from trigger point to trigger point, e.g., on the skin.

Finally, in addition to position detection and storage, an appliance setting of the electrotherapy appliance, in particular the pulse control of the electrotherapy appliance, can also be detected and logged. Advantageously, an appliance setting can be detected and logged for each detected position. This is particularly advantageous in order to carry out an optimum appliance setting on a patient-specific basis from several therapy protocols.

Accordingly, the electrotherapy appliance according to the invention may comprise a control device equipped to perform the aforementioned method.

### BRIEF DESCRIPTION OF THE DRAWING FIGURES

The above-described properties, features, and advantages of the present invention, as well as the manner in which they are achieved, will become more understandable in connection with the following description of the exemplary embodiments, which will be explained in more detail in connection with the drawings, wherein:

FIG. 1 shows a schematic diagram of an electrotherapy appliance,

FIG. 2 shows a schematic representation of a treatment head of the electrotherapy appliance of FIG. 1,

FIGS. 3a and 3b show a schematic representation of a protocol of a therapy with the electrotherapy appliance of FIG. 1, and

FIGS. 4a and 4b show a schematic representation of a protocol of an alternative therapy with the electrotherapy appliance of FIG. 1.

In the figures, identical technical elements are given the same reference signs and described only once. The figures are purely schematic and, above all, do not reflect the actual geometric relationships.

### DETAILED DESCRIPTION

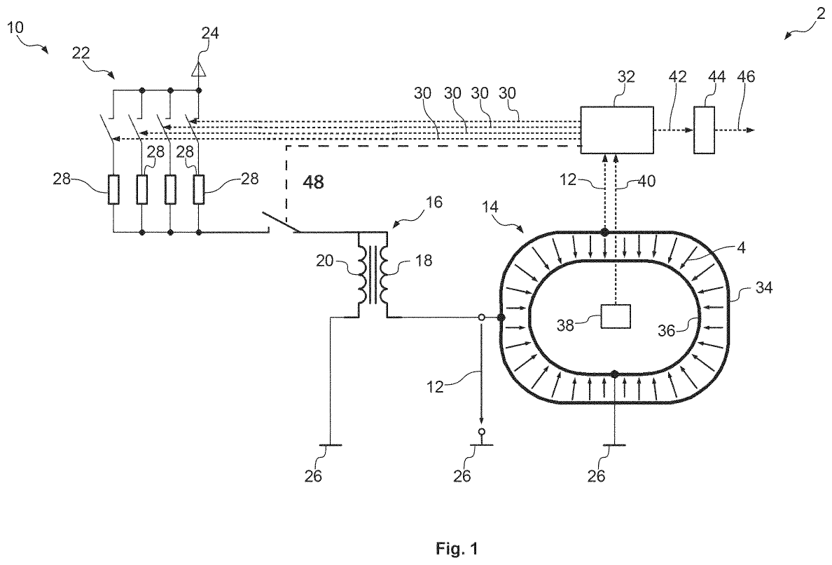

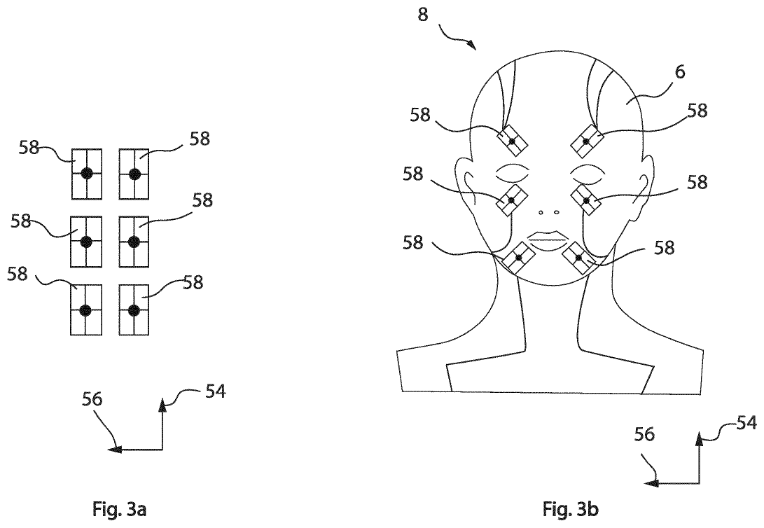

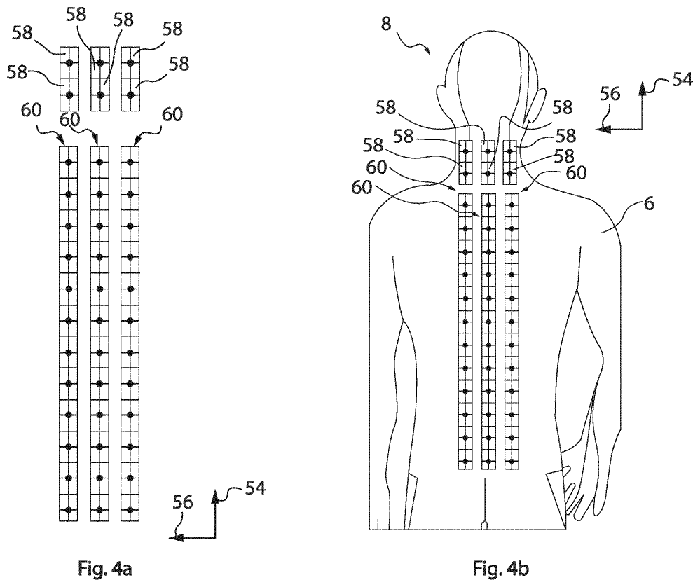

Reference is made to FIG. 1, which shows a schematic diagram of an electrotherapy appliance 2 for applying a therapeutic electrical pulse 4 to the skin 6 of a patient 8 shown in FIGS. 3b and 4b. The therapeutic pulse 4 is delivered in the form of an electric field, which is indicated by its field lines in FIG. 1.

To generate the therapeutic pulse 4 in the form of the electric field, the electrotherapy appliance 2 comprises a pulse source 10 for outputting a pulse voltage 12. In the present embodiment, the pulse source 10 comprises a resonant circuit formed by an output capacitor 14 outputting the therapeutic pulse 4 and a transformer 16 formed by a secondary coil 18 connected to the output capacitor 14 and a primary coil 20 feeding the secondary coil 18.

The primary coil 20 is connected between a supply potential 24 and ground 26 via a variable series resistor 22. The series resistor is formed by a parallel circuit of individual series resistors 28, which can be connected in a dedicated manner via control signals 30 from a control device 32 for power matching. In this way, the primary coil 20 can be energized with different currents so that different magnetic fields can be established in the secondary coil 18. If the current through the primary coil 20 is interrupted by disconnecting all the individual series resistors 28, the magnetic field in the secondary coil 18 suddenly decays and causes the oscillating circuit consisting of the secondary coil 18 and the output capacitor 14 to oscillate. The voltage thus excited in the output capacitor 14 generates the therapeutic pulse 4.

In principle, the output capacitor 14 can be of any design, such as stub lines. In the present embodiment, the output capacitor 14 has a first capacitor electrode 34 connected to the secondary coil 18. The first capacitor electrode 34 is of annular design and is also connected to the control device 32 for sensing the pulse voltage 12. The output capacitor 14 further includes a second capacitor electrode 36 of annular configuration which is concentrically arranged within the first capacitor electrode 34. The second capacitor electrode 36 is connected to ground 26, such that the secondary circuit connected to the transformer 16 via the secondary coil 18 is closed. Optionally or additionally, reversing the polarity of the output electrodes, referred to here as the capacitor electrodes, may result in an inverse alignment of the electric field, which would result in an inverted therapeutic pulse. Then the output electrode 34 is connected to ground and the output electrode 36 is connected to the secondary coil 18.

To use the electrotherapy appliance 2, the output capacitor 14 is placed on the skin 6 of the patient 8 and by driving one or more individual series resistors 28, the secondary coil 18 is charged via the primary coil 20. By disconnecting all of the individual series resistors 28 from the primary coil 20, the therapeutic pulse 4 is entered into the skin 6 of the patient 8 in the manner already explained. All control of this process may be accomplished by the control device 32.

In a preferred alternative, the individual resistors 28 are used to set the charging current for the primary coil 18. A further switch 48 can be provided on the common line to the primary coil which has two functions.

A first function is to isolate the control electronics 32 from the high pulse voltage of the primary 20 and secondary coil 18. Since the resistors 28 are very low impedance it would lead to immediate destruction of the control electronics, unless one designs all the control electronics for the high voltage range (>60 Vdc).

A second function is to provide centralized timed pulse control. By using this additional switch, a simple and always temporally constant pulse control is possible, independent of the position of the switches 18 for the current to the primary coil 20.

In order to document or log the therapy, the pulse voltage 12 applied to the output capacitor 14 can be recorded by the control device 32. The pulse voltage 12 can be used to derive measured values that describe a condition of the skin. For example, it is possible to determine from the pulse voltage 12 the resonant frequency and, via this, the capacitance of the skin 6, which in turn depends on how moist or dry the skin 6 is.

An effect of the therapy depends, among other things, on the energy with which the pulse voltage 12 is applied to the skin 6. The aforementioned therapy protocol should help to recognize the success of the therapy and to avoid unnecessary application of pulse voltage 12 to the skin 6.

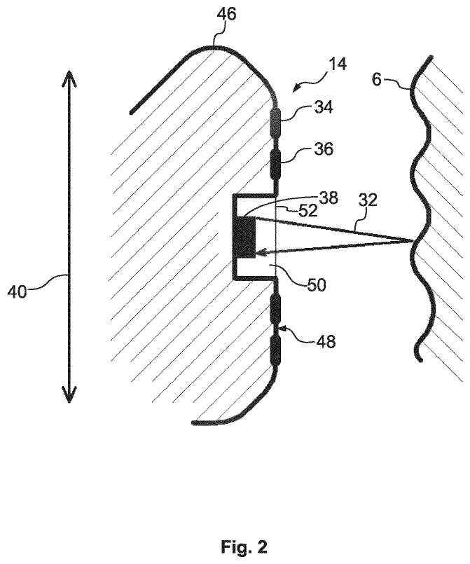

To provide the most accurate therapy protocol, the electrotherapy appliance 2 has a position sensor 38 for sensing a position 40 of the output capacitor 18 on the skin 6 during delivery of the pulse voltage 12, as indicated in FIG. 2.

The detected position 40, together with the measured value(s) describing the skin 6 at the detected position 40, can be combined in the control device 32 to form a data packet 42 and sent as a transmission signal 46 via an interface 44. The interface 44 is to be selected depending on the transmission technology to be used, such as Ethernet, Wireless Local Area Network, Nearfield Communication or Infrared. Transmission via Bluetooth Low Energy is particularly suitable as a transmission technology that saves space, energy, and bandwidth.

Reference is made to FIG. 2, which shows a schematic diagram of a treatment head 46 of the electrotherapy appliance 2 of FIG. 1, on which the output capacitor 18 and the position sensor 38 are arranged.

The treatment head 46 has a support surface 48 on which the output capacitor 18 is arranged. The support surface 48 can be placed on the skin 6 parallel to it so that the output capacitor 14 is arranged between the treatment head 46 and the skin 6.

Within the second annularly formed capacitor electrode 36—concentrically thereto in FIG. 2—the position sensor 38 is arranged in a recess 50, or also called cavity or indentation. The recess 50 is closed by a transparent cover 52.

Through the transparent cover 52, the position sensor 38 illuminates the skin 6 with a monochromatic or multicolored light 32. The skin 6 reflects the light 32 and partially reflects it back to the position sensor 38. The position sensor 38 picks up the reflected light 32 with a photosensor matrix and evaluates the resulting image. Depending on whether and how the image thus formed changes, a change in the position 40 on the skin 6 can be detected therefrom by the position sensor 38. In this way, the position 40 of the treatment head 46 and thus of the output capacitor 14 on the skin 6 can be detected incrementally. This measurement principle of the position 40 is well known from the computer mouse, and therefore need not be further discussed herein.

Finally, FIGS. 3 and 4 illustrate the creation of exemplary therapy protocols.

In FIGS. 3a and 3b, the head of patient 8 is shown. For the sake of simplicity, it should be regarded as rotationally symmetrical about the longitudinal axis of the body.

In this way, the patient 8 can be viewed in a cylindrical coordinate system with a height direction 54 extending in the direction of the longitudinal axis of the body and an angular direction 56 extending tangentially around the longitudinal axis of the body. The distance of the skin surface of the patient 8 from the longitudinal axis of the body is assumed to be constant for the sake of simplicity.

These assumptions are justified because the previously described photosensing position sensor 38 only detects the movement of the output capacitor 40 on the skin 6 in two dimensions anyway and fades out a radial movement of the output capacitor 40.

There are six therapy points 58 on the skin 6 in the area of the patient's head to which output capacitor 40 is to apply the therapeutic pulse 4 at a predetermined strength.

The therapist moves the treatment head 46 over the head of the patient 8 without setting it down and switches on the therapeutic pulse only at the individual therapy points 58. In this way, on the one hand the strength of the registered therapeutic pulse 4, but also the above-mentioned measured value, for example in the form of the resonant frequency, can be recorded and sent in the transmission signal 46 for further evaluation.

Here, the determination of position on the back of a patient with a two-dimensional position determination is more exact than in three-dimensional space, for example on the head. With this type of protocol, the method runs in such a way that the temporal sequence of the skin contacts establishes the assignment to the therapy points (58a-58f).

Example: The application begins with point 58a. The treatment head 40 is placed on point 58a, the control electronics optically detect the skin contact, begin with the therapeutic pulse and measure the skin and tissue reaction via the change in the parameters of the resonant frequency (bio-feedback). After a defined event (temporal or based on certain resonance parameters), an automatic entry is made in the digital therapy protocol via transmission signal 46. The user is prompted to detach the treatment head 40 from the skin and move to the next therapy point, e.g., 58b. The procedure repeats itself with the number of therapy points (here in the face 6) and with the number of therapy runs to be performed.

However, it is possible to extend the position determination by a third dimension or to determine the device orientation, which is taken into account when assessing the accuracy. This can be achieved by a suitable sensor, such as a gyroscope. Such sensors are already used extensively in cell phone applications.

This can be implemented in the same way on the back of patient 8 shown in FIGS. 4a and 4b.

As on the head, six therapy points are also located on the back, here in the area of the neck, which can also be treated in the same way. The application for these therapy points is explained above.

In addition, three therapy paths 60 also run on the back, via which the therapist can move the treatment head 46 without switching off the therapeutic pulse 4. The data on these therapy paths 60 can be continuously acquired and sent in the transmit signal 46 at specific local sampling distances, depending on the local resolution accuracy of the position sensor 38. In particular, in-plane position detection can be performed as follows: Via the dx and dy changes, starting from a predefined therapy starting point (detection of skin contact), the path of the treatment head 46 can be tracked, an automatic entry in the digital therapy protocol can be initiated via transmission signal 46 after defined intervals, or a deviation from the therapy path can be signaled to the therapist by sound or light signal (monitoring function). It should be noted that therapy is performed according to a pattern or rules which are specified, tracked, and monitored digitally (graphic display on a tablet or PC).

Optionally or additionally, a G-sensor can be integrated, which determines the accelerations in all 3 axes. The information of this sensor is used to support and validate the movement of the optical displacement sensor. The sole use of the information of a G-sensor is not sufficient to describe a path, because at a uniform velocity (acceleration=0) no motion information is available. However, the beginning and the end as well as the direction of a movement can be determined very well, also in space, i.e., on the X, Y and Z axis. The determination of the path from A to B is carried out as described by the dx/dy displacement sensor.

It is also thus possible to at least interpolate the relative movement from one therapy point to the next without skin contact.

Furthermore, the electrotherapy appliance according to the invention can have one or more distance sensors, e.g., laser sensors, which measures skin irregularities and takes these into account during signal processing.

Further, the electrotherapy appliance according to the invention may include one or more force or pressure sensors to measure the force of the imprint of the treatment head on the skin surface and to guide the therapist to provide a consistent and effective tactile treatment by light and/or audible signals.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-21. (canceled)

22. An electrotherapy appliance configured to introduce a therapeutic electrical pulse into skin of a patient, the electrotherapy appliance comprising:
a pulse source configured to output a pulse voltage;
an output capacitor, which is placeable on the skin of the patient and is fitted with a first capacitor electrode connected to a first terminal of the pulse source and fitted with a second capacitor electrode connected to a second terminal of the pulse source, in order to convert the pulse voltage into the therapeutic pulse; and
a position sensor configured to detect a position of the output capacitor on the skin while the therapeutic pulse is being delivered.

23. The electrotherapy appliance of claim 22, wherein the position sensor is a dx−dy displacement sensor.

24. The electrotherapy appliance of claim 22, wherein the position sensor is an optical displacement sensor.

25. The electrotherapy appliance of claim 22, wherein the pulse source comprises an oscillating circuit with the output capacitor and a magnetically chargeable coil, which is part of a transformer.

26. The electrotherapy appliance of claim 22, further comprising:
a transmitting interface configured to transmit the position of the output capacitor to a data processing device.

27. The electrotherapy appliance of claim 26, wherein the transmitting interface is configured to transmit the position of the output capacitor together with a measured value describing the skin at the detected position.

28. The electrotherapy appliance of claim 22, wherein the position sensor is configured to incrementally detect the position of the output capacitor on the skin.

29. The electrotherapy appliance of claim 28, wherein the position sensor, in order to incrementally detect the position, is configured to capture an image of the skin at regular intervals and to compare the captured image with a previously captured image of the skin.

30. The electrotherapy appliance of claim 22, wherein the first and second capacitor electrodes are closed loops arranged concentrically to one another.

31. The electrotherapy appliance of claim 30, wherein the position sensor is arranged within an inner loop of the closed loops arranged concentrically to each other.

32. The electrotherapy appliance of claim 22, wherein the output capacitor is held on a treatment head having a recess accommodating the position sensor.

33. The electrotherapy appliance of claim 32, wherein the recess is closed with a transparent cover.

34. The electrotherapy appliance of claim 22, further comprising:
an acceleration sensor configured to evaluate the position detection of the position sensor.

35. The electrotherapy appliance of claim 22, further comprising:
a control device configured to detect a movement of the electrotherapy appliance on the skin with respect to a predetermined starting point, so that the positions are detected as relative positions with respect to the starting position by the position sensor, wherein the control device is configured to transmit the positions to an interface of the electrotherapy appliance for logging.

36. The electrotherapy appliance of claim 35, wherein the control device is configured to generate a data packet comprising the detected position together with a measured value determined at the skin at the detected position.

37. The electrotherapy appliance of claim 36, wherein the measured value comprises a resonant frequency of the oscillating circuit in the form of a first pulse time, a number of zero crossings, or capacitance of the skin at the detected position.

38. A method for establishing a therapy protocol using electrotherapy appliance comprising a pulse source configured to output a pulse voltage, an output capacitor, which is placeable on the skin of the patient and is fitted with a first capacitor electrode connected to a first terminal of the pulse source and fitted with a second capacitor electrode connected to a second terminal of the pulse source, in order to convert the pulse voltage into the therapeutic pulse, and a position sensor configured to detect a position of the output capacitor on the skin while the therapeutic pulse is being delivered, the method comprising:
detecting a movement of the electrotherapy appliance on the skin relative to a predetermined starting point so that the positions are detected as relative positions relative to the starting position by the position sensor by ΔX and ΔY coordinates; and
logging the detected positions.

39. The method of claim 38, wherein, in addition to the respective position, a measured value determined by the electrotherapy appliance about a state of the skin at the time of the position measurement.

40. The method of claim 39, wherein the measured value is determined from a pulse voltage as a value of a resonant frequency or as a value of the capacitance of the skin.

41. The method of claim 38, wherein, in addition to the position, a detection and logging of a device setting of the electrotherapy appliance is performed, wherein the device setting is a pulse control of the electrotherapy appliance as a therapy program and energy level.

42. The method of claim 41, wherein for each detected position a device setting is detected and logged.
